(19) United States
(12) Patent Application Publication
(10) Pub. No.: US 2026/0265168 A1
(43) Pub. Date: Sep. 10, 2026

(54) METHOD FOR PRODUCING A MUSK FRAGRANCE INTERMEDIATE USING COPPER CATALYSTS

(71) Applicant: International Flavors & Fragrances Inc., New York, NY (US)

(72) Inventors: Jordi Pastor-Folch, Benicarlo (ES); Carlos López-Cruz, Benicarlo (ES); Jorge Sánchez-Quesada, Benicarlo (ES); Antonio Leyva-Pérez, Bétera (ES); Francisco Garnes-Portolés, Altura (Castellón) (ES)

(21) Appl. No.: 19/167,467

(22) PCT Filed: Mar. 27, 2024
(86) PCT No.: PCT/US2024/021709

§ 371 Date: Sep. 22, 2025

(30) Foreign Application Priority Data
Mar. 30, 2023 (EP) 2338230.0

(51) Int. Cl. C07C 41/03; C11B 9/00

(52) U.S. Cl. CPC C07C 41/03; C11B 9/0007; C07C 2601/14

(57) ABSTRACT

Disclosed is a method for producing a musk fragrance intermediate by reacting an alcohol with an epoxide in the presence of CuCl₂ or CuBr₂ or both as catalyst.

### BACKGROUND

In the fragrance industry there is a constant demand for compounds having pleasing odor properties. Such compounds extend a perfumer's palette and result in greater product diversity for consumers. In particular, there is demand for compounds that have musk odor characteristics. Such compounds are highly esteemed in perfumery and are perhaps some of the most versatile and common compounds found in fragrance compositions. Exemplary musk fragrances include, e.g., Helvetolide® (Firmenich), Romandolide® (Firmenich), Serenolide (Givaudan), and Appelide (International Flavors & Fragrances Inc.) and derivatives thereof. These, and other musk fragrances are described in, e.g., WO 2002/096852 A1 to Givaudan SA; WO 2004/050595 A1 to Givaudan SA; WO 2004/050602 A1 to Givaudan SA; WO 2005/108534 A1 to Givaudan SA; WO 2011/29895 A2 to Givaudan SA; U.S. Pat. No. 5,166,412 A to Firmenich SA; WO 2000/014051 A1 to Firmenich SA; WO 2009/034510 A2 to Firmenich SA; U.S. Pat. No. 6,384,269 B1 to Firmenich SA; WO 2005/01222 2 A1 and EP 1492759 B1 to Symrise AG; US 2004/053811 A1 to International Flavors & Fragrances Inc.; and WO 2019/124533 A1 to Takasago International Corp.

Epoxides are versatile intermediates in organic transformations to products of interest, due to their availability and their capability to react with a large amount of nucleophiles such as water, alcohols, amines, hydrides or halides. In particular, the O-alkylation of alcohols with epoxides through ring opening reactions is a common route for the production of 6-alkoxyalcohols, which are compounds of interest to the pharmaceutical, solvents and fragrance industry.

On one hand, the key challenges for these reactions, are the tendency of epoxides, especially terminal epoxides, to polymerize, which will directly affect overall selectivity to the O-alkylation product. Thus, mild conditions are required in order for the reactions to work. Moreover, the higher reactivity of the reaction product towards a second O-alkylation with epoxides results in low performance (low selectivity) reactions.

In order to overcome this inherent limitation of the O-alkylation of alcohols, one can use a selective homogeneous catalyst, in which the catalyst and the reactants are homogeneously mixed in the reaction media. A catalyst, in general, allows to activate molecules and decrease energy barriers (activation energy), towards the formation of intermediate compound precursors of the desired product and away from intermediate compound precursors leading to the formation of side products. Thus, a catalyst accelerates reaction rates towards desired compounds vs undesired ones, thereby allowing reactions to be performed faster, under milder conditions and with better selectivity.

Nowadays, many methods to produce this type of compounds via O-alkylation reaction make use of homogeneous catalytic systems, such as ethylaluminum dichloride (EP1262474 A1), boron trifluoride diethyl ethearate (U.S. Pat. No. 5,166,412 A), methylaluminium dichloride (WO2004 050602 A1) or tin chloride (WO 2008/046239). Each of these methods presents several drawbacks when scaling up to industrial scale, as noted in some cases which will be further elaborated. For example, the methods use expensive and hazardous reagents or in some cases, there is the need to perform the reaction at low temperatures or there is the need to use high amounts of catalyst.

One of the most efficient methods makes use of SnCl₂, used often in stoichiometric amounts. In addition, this mentioned industrial alcohol O-alkylation processes operate at low conversion in diluted system. The combination of both allows for reducing the selectivity loss by formation of an intermediate adduct of the metal salt and the desired product. Then, the final product is recovered by means of downstream work-up steps involving washing with strong acid, typically HCl, which requires the use of glass-line reactor, separation of organic and aqueous streams and, finally, neutralization or organic stream with sodium hydroxide solution. Therefore, it is believed that current alcohol O-alkylation processes are typically low efficient processes which produce substantial amounts of organic solvents and aqueous waste due to required downstream workups.

Given the value of these musk fragrances, there remains a need for a cost effective, high yield and more sustainable friendly method for producing said compounds.

### SUMMARY OF THE INVENTION

A method for producing a musk fragrance intermediate (e.g., a musk fragrance intermediate of Formula (I), in particular demol) by reacting an alcohol (e.g., an alcohol of Formula (II), in particular cyclademol) with an epoxide (e.g., an epoxide of Formula (III), in particular isobutylene oxide) in the presence of CuCl₂ or CuBr₂ or both as catalyst thereby producing the musk fragrance intermediate. In some aspects, the method is carried out in the absence of a solvent, and is carried out, using a catalyst in sub-stoichiometric amounts, that is less than 1 mol of catalyst per 1 mol of main reactant, and at a reaction temperature in the range of 30-35° C., or is carried out at atmospheric pressure or in both the mentioned reaction temperature and the mentioned atmospheric pressure. Thus, the catalyst of this method, in one example, is employed in sub-stoichiometric amounts, i.e., the enzyme is used in an amount lower than the reactant, for example, not in a 1:1 ratio as the reactant.

### DETAILED DESCRIPTION OF THE INVENTION

The examples provided in the detailed description are merely examples and should not be used to limit the scope of the claims in any claim construction or interpretation.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, use of “a” or “an” are used to describe elements and components described herein. This is done merely for convenience and to give a general sense of the scope of the invention. This description should be read to include one or at least one and the singular also includes the plural unless it is obvious that it is meant otherwise.

Unless otherwise defined, all technical and scientific terms used herein have the same meaning as commonly understood by one of ordinary skill in the art to which this invention belongs. In case of conflict, the present specification, including definitions, will control. Although methods and materials similar or equivalent to those described herein can be used in the practice or testing of embodiments of the present invention, suitable methods and materials are described below. In addition, the materials, methods, and examples are illustrative only and not intended to be limiting.

When an amount, concentration, or other value or parameter is given as either a range, preferred range or a list of upper preferable values and/or lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. For example, when a range of “1 to 10” is recited, the recited range should be construed as including ranges “1 to 8,” “3 to 10,” “2 to 7,” “1.5 to 6,” “3.4 to 7.8,” “1 to 2 and 7-10,” “2 to 4 and 6 to 9,” “1 to 3.6 and 7.2 to 8.9,” “1-5 and 10,” “2 and 8 to 10,” “1.5-4 and 8,” and the like.

The present specification illustratively described herein suitably may be practiced in the absence of any element or elements, limitation or limitations that are not specifically disclosed herein. While compositions and methods are described herein in terms of “comprising” various components or steps, the compositions and methods also can “consist essentially of” or “consist of” the various components or steps, unless stated otherwise.

A person of ordinary skill in the art appreciates that some chemical compounds in this specification have chiral center, carbon-carbon double bond, and/or cyclic structure. Unless explicitly indicated, a chemical compound in this specification includes its stereoisomers, such as enantiomers and diastereomers.

Cyclademol is 1-(3,3-dimethylcyclohexyl) ethanol.

The musk fragrance Helvimor® is produced via a 2-step process from cyclademol (1-(3,3-dimethylcyclohexyl) ethanol) (Scheme 1). The key intermediate in this process is Demol (2-[1-(3,3-dimethylcyclohexyl) ethoxy]-2-methylpropan-1-ol), which has been synthesized from cyclademol using BF₃ (see EP 2200963 B1) or stoichiometric amounts of SnCl₂ as catalysts. Due to the use of high amounts of SnCl₂ (26 wt %), the yield per pass is very low (approximately 28 wt %). The present specification provides a simple, commercially feasible method for producing Demol using CuCl₂ or CuBr₂ or both as the catalyst. In addition to being inexpensive, these catalysts provide an increase in the production of Demol compared to the use of SnCl₂. Moreover, there is a significant reduction in the amount of solvent and catalyst used, as well as wastewater produced.

Accordingly, this specification discloses a method for producing a musk fragrance intermediate by reacting a suitable alcohol with an alkylating agent, in particular an epoxide, in the presence of CuCl₂ or CuBr₂ or both as catalyst thereby selectively achieving o-alkylation of the alcohol and producing the musk fragrance intermediate.

In particular aspects, this specification provides for the production of a musk fragrance intermediate of Formula (I):

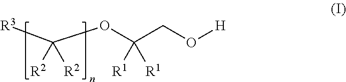

wherein
n represents 1 or 0,
each R¹, independently from each other, represents a hydrogen atom or a methyl or ethyl group, or the two R¹ taken together represent a (CH₂)ₘ group, m representing 3, 4, or 5;
each R², independently from each other, represents a hydrogen atom or a methyl or ethyl group, or the two R² taken together represent a (CH₂)ₘ group, m representing 3, 4, or 5; and
R³ represents a phenyl group optionally substituted, a saturated or unsaturated C₅-C₆ cyclic hydrocarbon moiety optionally substituted, or a CH(R⁴)₂, or R⁴CH═CR⁴ moiety, R⁴ representing a C₁-C₆ alkyl or alkenyl group optionally substituted.

According to particular aspects of the process as disclosed in this specification, the compound of Formula (I) is one in which n is 1, each R¹ is independently a hydrogen atom or methyl group, each R² is independently a hydrogen atom or methyl group, and R³ represents a phenyl group optionally substituted, a saturated or unsaturated C₅-C₆ cyclic hydrocarbon moiety optionally substituted, or a CH(R⁴)₂, or R⁴CH═CR⁴ moiety, R⁴ representing a C₁-C₆ alkyl or alkenyl group optionally substituted.

In some aspects, optional substituents of R³ are one, two or three C₁-C₃ alkyl groups, C₁-C₃ alkenyl groups, or C₁-C₃ alkoxy groups. In particular, optional substituents of R³ are one, two or three methyl or ethyl groups. Non-limiting typical examples of R³ groups include 3,3-dimethyl-cyclohexyl, 3,3-dimethylcyclohex-1-en-1-yl, 4-methyl-pent-2-en-2-yl, 5-methyl-cyclohex-3-en-1-yl, and 2-methyl-cyclohexyl. In particular aspects, the musk fragrance intermediate of Formula (I) is demol (2-[1-(3,3-dimethylcyclohexyl) ethoxy]-2-methylpropan-1-ol).

Alcohol. In one example, the alcohol used as the starting compound is an alcohol of Formula (II):

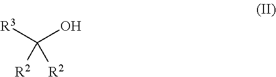

wherein each R², independently from each other, represents a hydrogen atom or a methyl or ethyl group, or the two R² taken together represent a (CH₂)ₘ group, m representing 3, 4, or 5; and
R³ represents a phenyl group optionally substituted, a saturated or unsaturated C₅-C₆ cyclic hydrocarbon moiety optionally substituted, or a CH(R⁴)₂, or R⁴CH═CR⁴ moiety, R⁴ representing a C₁-C₆ alkyl or alkenyl group optionally substituted. In particular aspects, the alcohol of Formula (II) is cyclademol (1-(3,3-dimethylcyclohexyl) ethanol).

Epoxides. Epoxides are cyclic ethers with three-membered cyclic rings composed of an oxygen atom attached to two adjacent carbon atoms. Epoxides of use in the method of this invention may have from 3 to 25 carbon atoms and one epoxy group. Exemplary epoxides include ethylene oxide, propylene oxide (1,2-propene oxide), butylene oxide (1,2-butene oxide), pentylene oxide (also known as 1,2-epoxypentane), hexylene oxide (also known as 1,2-epoxyhexane), octylene oxide (also known as 1,2-epoxyoctane), nonylene oxide (also known as 1,2-epoxynonane), decylene oxide (also known as 1,2-epoxydecane), isobutylene oxide, 4-methyl-1-pentylene oxide, and styrene oxide. In one example, the epoxide used in the method of this invention is an epoxide of Formula (III):

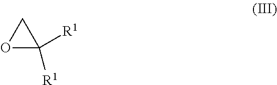

wherein each R¹, independently from each other, represents a hydrogen atom or a methyl or ethyl group, or the two R¹ taken together represent a (CH₂)ₘ group, m representing 3, 4, or 5. In particular aspects, the epoxide used in the process of the invention is isobutylene oxide.

In particular aspects, the reaction is carried out in the absence or substantial absence of a solvent (e.g., water and/or an organic solvent). For example, a substantial absence of a solvent can be no more than 5%, 2%, 1%, 0.5%, 0.25%, 0.1%, 0.05%, or 0.01% solvent by weight of the reaction mixture.

Accordingly, in some aspects, a musk fragrance intermediate of Formula (I) is prepared by reacting an alcohol of Formula (II) with an epoxide of Formula (III) in the presence of no more than 5%, 2%, 1%, 0.5%, 0.25%, 0.1%, 0.05%, or 0.01% solvent by weight of the reaction mixture (including starting materials, products, byproducts, and catalysts). In particular aspects, demol is prepared by reacting cyclademol with isobutylene oxide in the presence of CuCl₂ or CuBr₂ or both in the presence of no more than 5%, 2%, 1%, 0.5%, 0.25%, 0.1%, 0.05%, or 0.01% solvent by weight of the reaction mixture. In conventional methods for preparing demol, solvent can account for about 10-25 wt % of the total reaction mixture. Accordingly, the absence or substantial absence of solvent is a significant improvement in the preparation of this musk fragrance intermediate.

The reaction temperature at which a musk fragrance intermediate of Formula (I) is prepared, that is, the reaction temperature in the methods of this specification, is ideally between 20° C. and 70° C., or more preferably in the range of between 30° C. and 35° C. In a particular aspect, demol is prepared by reacting cyclademol with isobutylene oxide in the presence of CuCl₂ or CuBr₂ or both at a reaction temperature in the range of between 20° C. and 70° C., or more preferably in the range of between 30° C. and 35° C. Ideally the reaction is carried out at atmospheric pressure in batch or semi-batch mode. However, in some cases, the reaction may be carried out under reduced pressures conditions, e.g., between 0.5 and 100 mbar in batch mode or semi-batch mode.

Advantageously, as compared to the conventional homogeneous catalyst SnCl₂, either one of CuCl₂ or CuBr₂ or both can be used at a reduced amount in the O-alkylation of an alcohol to a musk fragrance intermediate. In some aspects, the method of this specification is carried out in the presence of a catalyst, and the catalyst comprises, consists essentially of, or consists of CuCl₂ or CuBr₂ or both. In some aspects, the CuCl₂ or CuBr₂ catalyst or both catalysts can be dry (e.g., in anhydrous form) or in hydrated form. In some aspects, the CuCl₂ or CuBr₂ or both catalyst is not loaded on a catalyst support (e.g., zeolite or activated carbon). In some aspects, the method of this specification is carried out in a reaction zone, and the CuCl₂ or CuBr₂ or both is or are fed into the reaction zone is not in a form of a complex with a ligand. In some aspects, the catalyst is CuCl₂, CuBr₂, or a mixture of CuCl₂ and CuBr₂. In some aspects, the catalyst is CuCl₂.

In some aspects, the mole ratio (also expressed herein as equiv, or Eq.) of catalyst to alcohol (e.g., cyclademol) is in the range of 0.02 to 0.2, or more preferably in the range of 0.04 to 0.10, or most preferably in the range of 0.06 to 0.08. The foregoing ranges are examples of a catalyst used in a sub-stochiometric amounts. In other aspects, the mole ratio of epoxide (e.g., isobutylene oxide) to alcohol (e.g., cyclademol) is in the range of 0.5 to 2.0, more preferably 0.8 to 1.4, or most preferably 1.0 to 1.2. In some aspects, the alcohol (e.g., cyclademol) and the catalyst are fed into a reaction zone, followed by the addition of epoxide (e.g., isobutylene oxide) into the rection zone at the reaction temperature continuously or in portions (e.g., in two or more portions). In other aspects, the amount of catalyst is less than 20 wt % of the reaction mixture, less than 18 wt % of the reaction mixture, less than 15 wt % of the reaction mixture, less than 10 wt % of the reaction mixture, or less than 8 wt % of the reaction mixture. Preferably, the amount of catalyst is about 5 wt % of the reaction mixture.

The present method provides the advantage of overall improvements in yield and process productivity, including a decrease in the amount of catalyst, decrease in the reaction time, and increase in the percent conversion as compared to using SnCl₂. Moreover, in some aspects, the reaction does not require the use of a solvent. As such, the present reaction provides for reduced wastewater production and manufacturing costs associated with the preparation of musk fragrance intermediates as compared to using a homogeneous catalyst SnCl₂.

The following describes some examples.

Demol. In one example, the musk fragrance intermediate is 2-((S)-1-((R)-3,3-dimethylcyclohexyl) ethoxy)-2-methylpropan-1-ol (Demol).

### Example 1: Conventional Process for Synthesizing Helvimor from Cyclademol Using SnCl₂ (Scheme I)

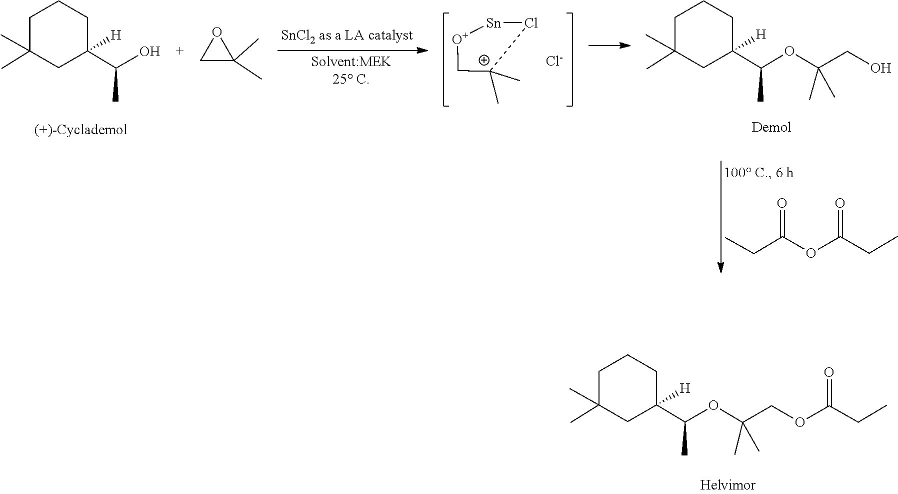

In general, cyclademol (1 eq), catalyst (0.24 eq) and other reagents (solvent, internal standard, etc.) are loaded in the reactor. When the desired temperature is reached (35° C.), isobutylene oxide (0.84 eq) addition starts. Samples are taken, analyzed by gas chromatography and when the reaction is finished, crude product is directly quenched with acid hydrolysis using diluted hydrochloric acid. The product is washed with water and neutralized with diluted sodium hydroxide. The molar yield of Demol is 23%. The organic crude product is distilled with a fractional column in order to recover unreacted cyclademol and Demol (95% purity).

### Example 2: Screening of Catalysts

Various catalysts were tested for their ability to catalyze conversion of cyclademol and isobutylene oxide to demol. For these reactions, cyclademol (1 eq, 1-15 g), catalyst (0.02-0.26 eq) and other reagents (solvent, internal standard, etc.) were loaded in the reactor (Table 1). When the desired temperature (0-35° C.) was reached, isobutylene oxide addition (0.75-1.60 eq) started (Table 1). Samples were periodically taken and analyzed by gas chromatography. When the reaction was finished, crude product was directly quenched with acid hydrolysis using diluted hydrochloric acid. The product was washed with water and neutralized with diluted sodium hydroxide. Based on, inter alia, percent conversion, yield, and ease of use, CuCl₂ and CuBr₂ were identified as suitable substitutes for SnCl₂ in the synthesis of demol.

TABLE 1

| | | Eq. | | | % Conversion | % Molar |
|---|---|---|---|---|---|---|
| Ex. | Catalyst | catalyst¹ | Eq IBO² | Solvent | Cyclademol | Yield Demol |
| 1 | SnCl₂ | 0.24 | 0.84 | MEK | 28 | 23 |
| 2 | CuCl₂ | 0.15 | 1.00 | — | 36 | 30 |
| 3 | CuCl₂ | 0.15 | 1.60 | — | 63 | 36 |
| 4 | BF₃ diethyl ethearate | 0.02 | 1.00 | — | 17 | 16 |
| 5 | Amberlyst 35 dry | 0.20 | 0.75 | — | 10 | 2 |
| 6 | CuCl | 0.15 | 0.75 | — | 1 | 0 |
| 7 | SnCl₄ | 0.26 | 0.75 | MEK | 16 | 2 |
| 8 | Bu₃SnOMe | 0.26 | 0.75 | MEK | 12 | 0 |
| 9 | Ti(Oct)₄ | 0.26 | 0.75 | MEK | 30 | 0 |
| 10 | SiO₂•SO₃ | 0.26 | 0.75 | MEK | 28 | 0 |
| 11 | FeCl₃ | 0.26 | 0.75 | MEK | 32 | 4 |
| 12 | Fe(NO₃)₃ | 0.26 | 0.75 | MEK | 57 | 6 |
| 13 | Fe₂(SO₄)₃ | 0.26 | 0.75 | MEK | 26 | 4 |
| 14 | Fe(acac)₃ | 0.26 | 0.75 | MEK | 0 | 0 |
| 15 | Fe₂O₃ | 0.26 | 0.75 | MEK | 3 | 0 |
| 16 | K₄FeCN₆ | 0.26 | 0.75 | MEK | 24 | 0 |
| 17 | CuBr₂ | 0.26 | 0.75 | MEK | 48 | 16 |
| 18 | CuBr₂ | 0.10 | 1.60 | — | 63 | 39 |
| 19 | CuBr | 0.26 | 0.75 | MEK | 8 | 0 |
| 20 | CuCO₃ | 0.26 | 0.75 | MEK | 0 | 0 |
| 21 | CuCN | 0.26 | 0.75 | MEK | 0 | 0 |
| 22 | Cu(acetate)₂ | 0.26 | 0.75 | MEK | 0 | 0 |
| 23 | Cu(acac)₂ | 0.26 | 0.75 | MEK | 0 | 0 |
| 24 | Cu(SO₃CF₃)₂ | 0.26 | 0.75 | MEK | 26 | 6 |
| 25 | Cu(CO₂CF₃)₂ | 0.26 | 0.75 | MEK | 42 | 9 |
| 26 | CuSO₄ | 0.26 | 0.75 | MEK | 0 | 0 |
| 27 | Cu(NO₃)₂ | 0.26 | 0.75 | MEK | 25 | 4 |
| 28 | CuO/SiO₂ (12 wt %) | 0.26 | 0.75 | MEK | 13 | 1 |
| 29 | NaX•CuBr₂ (5 wt %) | 0.26 | 0.75 | MEK | 3 | 0 |
| 30 | NaX•CuBr₂ (10 wt %) | 0.26 | 0.75 | MEK | 4 | 0 |

Ti(Oct)₄, Titanium tetraoctanate;
X, zeolite X;
MEK, Methyl Ethyl Ketone
¹Eq. Catalyst, Catalyst equivalent to cyclademol (i.e., the mole ratio of catalyst to cyclademol).
²Eq. IBO, Isobutylene Oxide equivalent to cyclademol (i.e., the mole ratio of isobutylene oxide to cyclademol).

### Example 3: Synthesis of Demol Using CuBr₂

CuBr₂ was further tested for its ability to catalyze the conversion of cyclademol and isobutylene oxide to demol under various process conditions increasing scale of experiments. For these reactions, cyclademol (1 eq, 100-300 g), catalyst were loaded in the reactor. When the desired temperature (35° C.) was reached, isobutylene oxide addition started. Samples were periodically taken and analyzed by gas chromatography. When the reaction was finished, crude product was directly quenched with acid hydrolysis using diluted hydrochloric acid. The product was washed with water and neutralized with diluted sodium hydroxide.

In these tests, catalyst equivalents, isobutylene oxide equivalents, and distribution of isobutylene oxide addition (Table 2) were adjusted.

TABLE 2

| | | Eq. | | Reaction | % Conversion | % Molar Yield |
|---|---|---|---|---|---|---|
| Ex. | Catalyst | Catalyst¹ | Eq. IBO² | time (h)³ | Cyclademol | Demol |
| 1 | SnCl₂ | 0.24 | 0.84 | 30 | 28 | 23 |
| 31 | CuBr₂ | 0.06 | 0.75 | 48 | 31 | 25 |
| 32 | CuBr₂ | 0.10 | 0.75 | 46 | 29 | 24 |
| 33 | CuBr₂ | 0.15 | 1.60 | 46 | 60 | 31 |
| 34 | CuBr₂ | 0.06 | 1.60 | 48 | 35 | 28 |
| 35 | CuBr₂ | 0.15 | 0.80 + 0.80 | 42 | 58 | 33 |

1 is benchmark processes.
¹Eq. Catalyst, Catalyst equivalent to cyclademol (i.e., the mole ratio of catalyst to cyclademol).
²Eq. IBO, Isobutylene Oxide equivalent to cyclademol (i.e., the mole ratio of isobutylene oxide to cyclademol). IBO added in a single addition or 2 additions (represented by “+”) at the indicated times.
³“Reaction time (h)” is the time at which the reaction was allowed to proceed.

The results of this analysis (Table 2) indicated that with lower catalyst loading (example 31), the conversion was limited to 31% with performance similar to conventional process 1 values (23 vs. 25% mol yield), although reaction time was increased (30 hours vs. 48 hours).

When evaluating the effect of increased catalyst loading (from 0.06 (example 31) to 0.10 (example 32) eq. catalyst to cyclademol) or isobutylene oxide loading (from 0.75 (example 31) to 1.6 (example 34) eq. isobutylene oxide to cyclademol), it was observed that the conversion values reached were very similar. In the case of increasing both catalyst and isobutylene oxide loading, conversion reached values between 55-60% (examples 33 and 35). These results indicated that it was beneficial to raise both parameters to achieve a higher conversion.

Notably, isobutylene oxide addition distribution seemed to have a positive effect on selectivity, especially for experiments with higher catalyst loading (0.15 eq, example 35). The isobutylene oxide addition was split into two portions. The first portion (50% of total amount) was added to favor dissolution of catalyst in crude and favor reaction rate, and the second portion (50% of total amount) was added slowly to avoid reagent accumulation and consequently by-product formation.

Based on molar yield, it was clear that process performance may be increased (from 23% to 33% mol). This was feasible based on an optimal loading ratio of catalyst (0.15 eq) and isobutylene oxide (1.6 eq) with respect to cyclademol, in addition to carrying out partial addition of isobutylene oxide at a low rate.

### Example 4: Synthesis of Demol Using CuCl₂

CuCl₂ was further tested for its ability to catalyze the conversion of cyclademol and isobutylene oxide to demol under various process conditions increasing scale of experiments. For these reactions, cyclademol (1 eq, 100-300 g), catalyst were loaded in the reactor. When the desired temperature (35° C.) was reached, isobutylene oxide addition started. Samples were periodically taken and analyzed by gas chromatography. When the reaction was finished, crude product was directly quenched with acid hydrolysis using diluted hydrochloric acid. The product was washed with water and neutralized with diluted sodium hydroxide.

In these tests, catalyst equivalents, isobutylene oxide equivalents, and distribution of isobutylene oxide addition (Table 3) were adjusted.

TABLE 3

| | | Eq. | | Reaction | % Conversion | % Molar Yield |
|---|---|---|---|---|---|---|
| Ex. | Catalyst | Catalyst¹ | Eq. IBO² | time (h)³ | Cyclademol | Demol |
| 36 | CuCl₂ | 0.15 | 0.80 + 0.80 | 46 | 56 | 35 |
| 37 | CuCl₂ | 0.15 | 1.20 | 28 | 53 | 28 |
| 38 | CuCl₂ | 0.15 | 0.60 + 0.60 | 30 | 49 | 33 |
| 39 | CuCl₂ | 0.06 | 1.00 | 20 | 42 | 32 |

¹Eq. Catalyst, Catalyst equivalent to cyclademol (i.e., the mole ratio of catalyst to cyclademol).
²Eq. IBO, Isobutylene Oxide equivalent to cyclademol (i.e., the mole ratio of isobutylene oxide to cyclademol). IBO added in a single addition or 2 additions (represented by “+”) at the indicated times.
³“Reaction time (h)” is the time at which the reaction was allowed to proceed.

Similar performance in terms of conversion rates and molar yield after quenching were observed when comparing CuCl₂ with CuBr₂ (Example 36 from Table 3 vs. Example 35).

Notably, the analysis of the results also indicated that it reduces both isobutylene oxide (Examples 37 & 38) and catalyst (Example 39) loadings with respect to CuBr₂ in order to achieve similar conversion (40-50%). When comparing time vs. cyclademol conversion, it was found that reaction times could be reduced to achieve higher conversion with CuCl₂ as catalyst.

### Example 5: Parameters for Synthesizing Demol Using CuCl₂

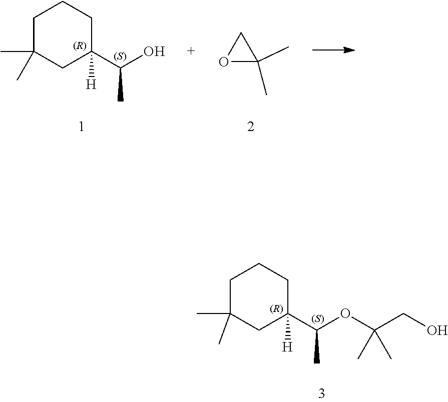

Cyclademol (1, 120 g, 1 eq.) and CuCl₂ (0.06-0.08 eq. with respect to cyclademol, i.e., the mole ratio of catalyst to cyclademol) and an internal standard (cyclademol+dodecane, 15 wt % with respect to cyclademol) were introduced into a reactor. The system was cooled to 5° C. and isobutylene oxide (2, 1.0-1.2 eq. with respect to cyclademol, i.e., the mole ratio of isobutylene oxide to cyclademol) was subsequently added to the reactor. After a short induction period, the reaction started, and the process temperature was adjusted to 30-35° C. All operations were carried out at atmospheric pressure. Samples were taken, quenched, and analyzed by gas chromatography during the reaction. When the reaction was finished, crude product was directly quenched via acid hydrolysis with diluted hydrochloric acid (10 wt %) by adding the crude reaction to the acid solution. The product was washed with water and neutralized with diluted sodium hydroxide (10 wt %). The organic crude product was distilled with a fractional column in order to recover unreacted cyclademol and Demol.

Demol (3, 2-[1-(3,3-dimethylcyclohexyl) ethoxy]-2-methylpropan-1-ol) was selectively produced (70-77 mol %) and the conversion achieved (40-45%) exceeded that of SnCl₂. Advantageously, the significant reduction in catalyst loading associated with the use of CuCl₂ as compared to SnCl₂ provided for reduced wastewater generation (Table 4).

TABLE 4

Catalyst

| Demol Reaction Conditions | SnCl₂ | CuCl₂ |
|---|---|---|
| Eq. Catalyst¹ | 0.24 | 0.06-0.08 |
| Eq. Isobutylene oxide² | 0.84 | 1.0-1.2 |
| Solvent | MEK | Solventless |
| Reaction Time (h) | 30 | 20 |
| % Conversion Cyclademol | 28 | 40-45 |
| % Molar Selectivity Demol | 82 | 70-77 |
| % Molar Yield Demol | 23 | 32 |
| kg Waste-water/kg Demol | 12-14 | 4-5 |

¹Eq. Catalyst, Catalyst equivalent to cyclademol (i.e., the mole ratio of catalyst to cyclademol).
²Eq. IBO, Isobutylene Oxide equivalent to cyclademol (i.e., the mole ratio of isobutylene oxide to cyclademol).
MEK, Methyl Ethyl Ketone.

Therefore, the use of CuCl₂ in the production of demol from cyclademol and isobutylene oxide is a significant improvement over the conventional use of SnCl₂ as catalyst, for example, improving the conversion rate by at least 40 percent and reducing the reaction time by 10 hours.

The range of recited numerical values disclosed in the specification includes values, e.g., +/−5-10% of the recited value, that a person of ordinary skill in the art would consider equivalent to the recited value, e.g., having the same function or result.

The claims are not limited by the preferred embodiments and examples but will cover many modifications and equivalents consistent with the written description as a whole.

1. A method for producing a musk fragrance intermediate comprising reacting an alcohol with an epoxide in the presence of a metal salt, preferably a copper salt and most preferably CuCl₂ or CuBr₂ or both as catalyst thereby producing a musk fragrance intermediate.

2. The method of claim 1, wherein the musk fragrance intermediate has the structure of Formula (I): 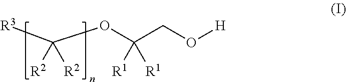
wherein
n represents 1 or 0,
each R¹, independently from each other, represents a hydrogen atom or a methyl or ethyl group, or the two R¹ taken together represent a (CH₂)ₘ group, wherein m represents 3, 4, or 5;
each R², independently from each other, represents a hydrogen atom or a methyl or ethyl group, or the two R² taken together represent a (CH₂)ₘ group, wherein m represents 3, 4, or 5; and
R³ represents a phenyl group optionally substituted, a saturated or unsaturated C₅-C₆ cyclic hydrocarbon moiety optionally substituted, a CH(R⁴)₂ moiety, or R⁴CH═CR⁴ moiety, wherein R⁴ represents a C₁-C₆ alkyl or alkenyl group optionally substituted.

3. The method of claim 1, wherein the alcohol has the structure of Formula (II): 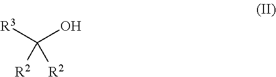
wherein
each R², independently from each other, represents a hydrogen atom or a methyl or ethyl group, or the two R² taken together represent a (CH₂)ₘ group, wherein m represents 3, 4, or 5; and
R³ represents a phenyl group optionally substituted, a saturated or unsaturated C₅-C₆ cyclic hydrocarbon moiety optionally substituted, a CH(R⁴)₂ moiety, or R⁴CH═CR⁴ moiety, wherein R⁴ represents a C₁-C₆ alkyl or alkenyl group optionally substituted.

4. The method of claim 1, wherein the epoxide has the structure of Formula (III): 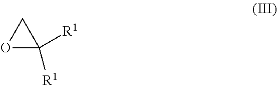
wherein
each R¹, independently from each other, represents a hydrogen atom or a methyl or ethyl group, or the two R¹ taken together represent a (CH₂)ₘ group, wherein m represents 3, 4, or 5.

5. The method of claim 1, wherein the musk fragrance intermediate is 3, 2-[1-(3,3-dimethylcyclohexyl) ethoxy]-2-methylpropan-1-ol.

6. The method of claim 1, wherein the alcohol is 1-(3,3-dimethylcyclohexyl) ethanol.

7. The method of claim 1, wherein the epoxide is isobutylene oxide.

8. The method of claim 1, wherein said method is carried out in the absence of a solvent.

9. The method of claim 1, wherein said method is carried out at a reaction temperature in the range of 10-100° C., preferably between 25-70° C., and most preferably between 30-45° C.

10. The method of claim 1, wherein said method is carried out atmospheric pressure and 10 bar, preferably between atmospheric pressure and 5 bar, most preferably at atmospheric pressure.

11. The method of claim 1, wherein said method is carried out in the presence of a copper salt as catalyst, most preferably CuBr₂ or CuCl₂, and most preferable, CuCl₂ as catalyst.

12. The method of claim 1, wherein said method is carried out in the presence of CuCl₂ in sub-stoichiometric amounts.
